(19) United States
(12) Patent Application Publication
(10) Pub. No.: US 2026/0284294 A1
(43) Pub. Date: Sep. 24, 2026

(54) Biological Material Processing Device

(71) Applicant: Push2Spin Technologies, Inc., Pittsburgh, PA (US)

(72) Inventors: Jeffrey A. Gusenoff, Pittsburgh, PA (US); Beth R. Gusenoff, Pittsburgh, PA (US); Denise Gottfried, Woodside, CA (US)

(73) Assignee: Push2Spin Technologies, Inc., Pittsburgh, PA (US)

(21) Appl. No.: 19/571,319

(22) Filed: Mar. 18, 2026

Related U.S. Application Data
(60) Provisional application No. 63/773,982, filed on Mar. 18, 2025.

(51) Int. Cl. A61M 1/00

(52) U.S. Cl. CPC A61M 1/892; A61M 1/895; A61M 2202/08

(57) ABSTRACT

A biological material processing apparatus may include a container configured to hold biological material and an entry port that permits the biological material to be introduced into the container. A rotatable core may be positioned within the container and may be rotatable relative to the container. An actuator may cause rotation of the core. An arm may be coupled to the core. The arm may be movable from a retracted configuration to an extended configuration during rotation of the core. The arm may be configured to engage and retain fibrous tissue or similar debris while the biological material is being processed within the container.

### CROSS-REFERENCE TO RELATED APPLICATIONS

This application claims the benefit of U.S. Provisional Patent Application No. 63/773,982 filed Mar. 18, 2025 entitled “Device and Methods of Filtering Tissue with Hand Driven Centrifugation”, which is incorporated by reference herein in its entirety.

### FIELD OF THE INVENTION

The present invention relates generally to devices and systems for processing biological material. More particularly, the present invention relates to devices configured to process biological material such as adipose tissue to separate, filter, or otherwise prepare the material for medical procedures including grafting and other therapeutic applications.

### BACKGROUND OF THE INVENTION

In a variety of medical procedures, biological material may be collected from a patient and subsequently processed before being reintroduced into the patient. For example, adipose tissue may be harvested from a patient using liposuction techniques and then processed prior to reinjection for cosmetic or reconstructive procedures such as facial augmentation, breast reconstruction, or treatment of soft tissue defects.

Processing harvested biological material may involve separating adipose tissue from other materials such as oil, blood, saline, and fibrous tissue. In many existing systems, the harvested material is transferred between multiple containers or syringes to facilitate separation and purification.

Some conventional systems may require multiple handling steps, may process material in discrete batches, or may provide limited capability for removing unwanted fibrous material or debris. Additionally, certain systems may require disconnecting tubing or transferring biological material between components during the processing process, which may increase procedure time or complexity. Accordingly, there remains a need for improved devices and systems capable of processing biological material in a controlled and efficient manner.

### BRIEF SUMMARY OF THE INVENTION

In one embodiment there is a biological material processing device comprising a container configured to receive biological material, an inlet port configured to receive the biological material into the container, a core disposed within the container and rotatable relative to the container, an actuator configured to rotate the core, and an arm coupled to the core. The arm may be movable from a retracted configuration to an extended configuration during rotation of the core. The arm may be configured to capture fibrous material within the biological material during rotation of the core. In some embodiments, at least a portion of the arm extends outward from a first position to a second position during rotation of the core and retracts to the first position as the core decelerates, optionally with the core including a recess such that at least a portion of the arm is in the recess in the first position and out of the recess in the second position. The arm may be rotationally fixed relative to the core about a first axis and rotatable relative to the core about a second axis offset from the first axis. The arm may comprise a base and a plurality of projections extending from the base, the projections spaced from each other along the base to capture fibrous strands within the biological material, optionally with the arm connected to the core by a hinge and comprising absorbent material configured to capture debris.

In some embodiments, the device further comprises a filter within the container, the filter positioned between the core and a sidewall of the container. The filter may be configured to separate liquid or oil components from the biological material. The filter may comprise a mesh having openings between approximately 10 micrometers and 500 micrometers. The filter may separate oil from adipose tissue.

Rotation of the core may process the biological material to separate solid components from liquid components. The device may further comprise a first outlet for removing the solid components from the container and a second outlet for removing the liquid components from the container. The device may include a valve configured to selectively prevent flow of material through at least one of the first and second outlets. The first outlet may be connected to tubing or a conduit for transferring the solid component to a patient. The solid component may comprise fat.

The device may further include a lid detachably coupled to the container. The lid may be configured to form a liquid tight seal with the container. The lid may include a locking mechanism that secures the lid to the container to maintain sterility. The lid may include an irrigation port for saline, antibiotics, or other fluids to be introduced into the container.

The actuator may be manually actuated and may comprise a push-to-spin mechanism. The device may further comprise a base coupleable with the container. The container may be rotatable relative to the base. The base may include a motor that rotates the container relative to the base. The actuator may rotation of the core while the container rotates relative to the base. In some embodiments, the container comprises a rotating biological processing chamber configured to maintain fluid communication with at least one stationary conduit during rotation.

In another embodiment, there is a tissue processing system comprising a container configured to receive biological material, a core disposed within the container, a first conduit configured to deliver biological material from a patient to the container, a second conduit configured to return biological material from the container to the patient, and a spinning mechanism configured to rotate at least one component of the tissue processing system. The biological material may circulate in a continuous closed loop between the patient and the container during rotation of the at least one component of the tissue processing system.

The system may further comprise a base configured to support the container. The base may contain a motor to allow the container to rotate relative to the base. The system may operate in a first mode in which the container rotates, a second mode in which the core rotates, or sequentially in both modes. The container may remain stationary while the core rotates. Biological material may be processed while circulating between the patient and the container such that aspiration and reinjection occur simultaneously.

The system may include an arm coupled to the core wherein the arm captures fibrous tissue during rotation. The arm may extend outward from a first position to a second position during rotation. The arm may retract as the rotation decelerates. The arm may rotate about a first axis and a second axis, offset from the first axis, simultaneously. The system may be configured for fat grafting procedures.

In another embodiment, there is a biological material processing device comprising a container configured to receive biological material, a core disposed within the container, a fluid port configured to communicate biological material between the container and an external conduit, and a base configured to secure the external conduit, wherein the core rotates relative to the fluid port while biological material flows through the fluid port. The base may hold the conduit in a fixed position and prevent rotation of the conduit. The conduit may remain stationary while the core rotates. The fluid port may remain fixed relative to external conduit while the core rotates. The base may include a rigid port adapter. The core may rotate about an axis that extends through at least a portion of the fluid port. The core may include deployable arms.

In another embodiment, there is a method of processing biological material comprising aspirating biological material from a patient into a container of a biological material processing device, rotating a component of the biological material processing device to separate components of the biological material, capturing impurities with deployable arms within the container, and removing processed tissue from the container. The method may include at least one of capturing fibrous tissue and circulating the biological material in a closed loop. The method may include rotating a core relative to the container or rotating the container relative to the core. The method may include extending at least a portion of the arm away from the core by centrifugal force. The method may include retracting the deployable arms by decelerating rotation of the core. The method may include reinjecting the processed tissue into the patient such that aspiration and reinjection occur simultaneously. The method may include at least one of filtering oil from adipose tissue and introducing saline or antibiotics into the container. The method may include rotating the component with a motor or a manually activated push-to-spin mechanism. The method may include separating oil from adipose tissue using a mesh filter and delivering processed adipose tissue for fat grafting.

### BRIEF DESCRIPTION OF THE SEVERAL VIEWS OF THE DRAWINGS

The foregoing summary, as well as the following detailed description of embodiments of the biological material processing device, will be better understood when read in conjunction with the appended drawings of at least one exemplary embodiment. It should be understood, however, that the invention is not limited to the precise arrangements and instrumentalities shown.

In the drawings:

FIG. 1 is a perspective view of a processing device in accordance with an exemplary embodiment of the present disclosure;

FIG. 2 is a front elevation view of a base that can be coupled to the processing device of FIG. 1 in accordance with an exemplary embodiment of the present disclosure;

FIG. 3 is a top plan view of the base of FIG. 2;

FIG. 4 is a front elevation, partial sectional view of a processing device in accordance with an exemplary embodiment of the present disclosure;

FIG. 5 is a front elevation view of a core in accordance with an exemplary embodiment of the present disclosure;

FIG. 6 is a perspective view of a processing device and a base in accordance with an exemplary embodiment of the present disclosure; and

FIG. 7 is an enlarged front elevation view of an adapter, coupling and core of FIG. 6.

### DETAILED DESCRIPTION OF THE EXEMPLARY EMBODIMENTS OF THE INVENTION

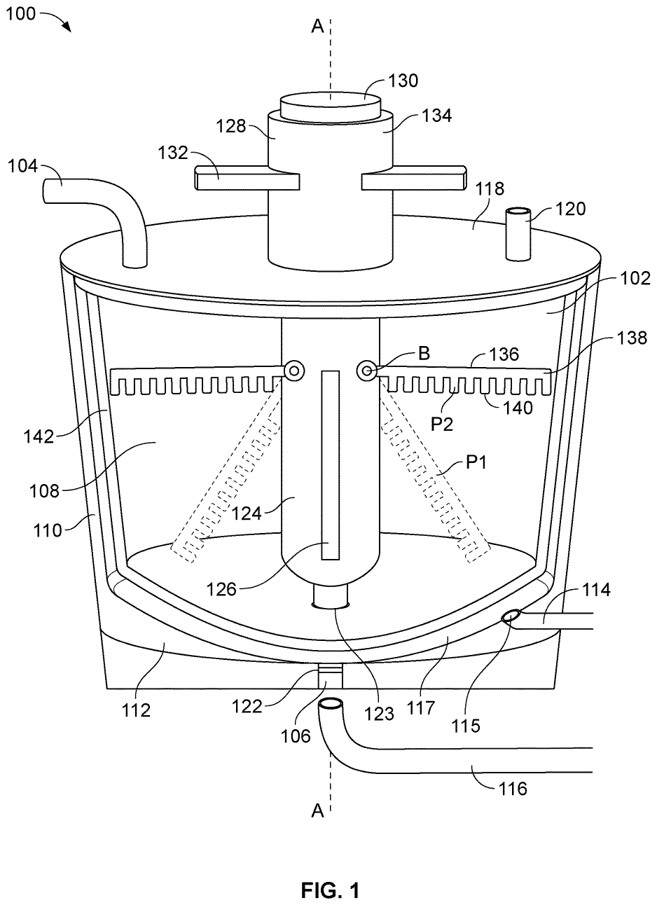

Referring to the drawings in detail, wherein like reference numerals indicate like elements throughout, there is shown in FIG. 1 a processing device or processor, generally designated 100, in accordance with an exemplary embodiment of the present invention. The processor 100 may be configured to receive, process, and deliver biological material. In one exemplary implementation, the processor 100 may be used to process adipose tissue harvested from a patient. The processor 100 may operate to separate one or more components of the biological material from other components of the biological material. For example, the processor 100 may separate oil, aqueous fluids, blood, debris, or other undesired fractions from adipose tissue to produce purified or concentrated adipose tissue suitable for grafting or reinjection into a patient. Although adipose tissue processing is described herein for purposes of illustration, it should be understood that the processor 100 may also be used to process other biological materials or tissue types such as bone marrow aspirate, platelet-rich plasma, stromal vascular fraction, and stem cell preparations.

In certain embodiments, the processor 100 may be part of a closed loop system that allows aspiration of biological material from a patient, processing of the biological material within the processor 100, and subsequent delivery of the processed biological material back to the patient. For example, adipose tissue may be harvested from a patient during a liposuction procedure and delivered to the processor 100 through tubing connected to an inlet port. After processing within the processor 100, the processed adipose tissue may be returned through tubing connected to an outlet port for reinjection into the patient. In some implementations, the processor 100 may allow simultaneous aspiration and reinjection such that biological material may circulate through the processor 100 while being processed.

The processor 100 may be configured to generate centrifugal forces within a processing chamber to facilitate separation of biological material components. In certain embodiments, centrifugal forces may be generated by rotating the entire processor or chamber, or alternatively by rotating internal components of the processor while external components remain stationary.

The processor 100 may be suitable for grafting procedures such as facial augmentation, fat grafting to the foot or hand, breast, or other cosmetic or reconstructive indications. The processor 100 may be manufactured from typical plastic and other materials, such as polymers, silicones, ceramics, metals, etc., commonly used for production of medical devices. The processor 100 may be disposable after a single use. Alternatively, the processor 100 may be reusable, either with the same patient, or if constructed of suitable materials able to withstand sterilization, with different patients. The processor 100 may allow for larger volume fat processing than existing fat processing syringes. For example, the processor 100 may be configured to process about 50 mL to 500 mL, about 500 mL to 1 L about 1 L to about 1.5 L, about 1.5 L to about 2 L, about 50 mL to about 2 L, about 50 L to about 1 L or about 1 L to about 2L, of biological material in a single processing cycle.

The processor 100 may include a container 102 having an inlet 104 and an outlet 106 such that biological material can be introduced into the container 102 through the inlet 104 and can exit the container 102 through the outlet 106. The container 102 may define a chamber 108 configured to receive biological material for processing. In operation, biological material may enter the chamber 108 through the inlet 104 and exit the chamber 108 through the outlet 106 after processing. The inlet 104 and outlet 106 may be configured to connect to tubing, conduits, or other fluid connectors to facilitate introduction and removal of biological material.

The container 102 may include a sidewall 110 that defines at least a portion of the chamber 108. The sidewall 110 may define a cylindrical shape, a frustoconical shape, or any other suitable geometry capable of containing biological material. In some embodiments, the sidewall 110 may be manufactured from a transparent or translucent material so that a user can observe biological material within the chamber 108 during processing. For example, a user may visually observe separation of adipose tissue from oil or other biological fluids during operation of the processor 100.

The container 102 may include a base or bottom wall 112 coupled to the sidewall 110. The bottom wall 112 may define a lower boundary of the chamber 108. In some embodiments, the bottom wall 112 and the sidewall 110 may be formed as a monolithic structure. In other embodiments, the bottom wall 112 and the sidewall 110 may be separate components that are joined together using adhesive, welding, fasteners, threaded connections, or other suitable joining techniques. The outlet 106 may include an opening 123 that extends through the bottom wall 112 or sidewall 110 such that material moves through the opening 123 and out of the outlet 106.

The container 102 may include a second outlet 114 configured to allow material to exit the chamber 108. In certain embodiments, the outlet 106 and the second outlet 114 may be arranged such that different fractions of biological material exit the chamber 108 through different outlets. For example, adipose tissue may exit through outlet 106 while oil or other biological fluids may exit through the second outlet 114. In some examples, one of the outlet 106 and the second outlet 114 may extend through the sidewall 110 while the other extends through the bottom wall 112. In other configurations, both outlets may extend through the sidewall 110 or both may extend through the bottom wall 112. The second outlet 114 may include an opening 115 disposed in at least one of the sidewall 110 and the bottom wall 112.

The processor 100 may include one or more valves 122 or other devices configured to selectively control flow of biological material through the outlet 106 and the second outlet 114. In some embodiments, each outlet may include an independently operable valve 122. The valve 122 may include a ball valve, check valve, flow restrictor, pinch valve, or other flow control device capable of regulating fluid flow from the container 102.

A lid 118 may be detachably coupled to the container 102. When coupled to the container 102, the lid 118 and container 102 may form a fluid-tight seal that maintains biological material within the chamber 108. In some embodiments, the lid 118 and container 102 may cooperate to maintain a sterile environment within the chamber 108. The processor 100 may include a locking mechanism configured to temporarily secure the lid 118 to the container 102. For example, the locking mechanism may include an interference fit between the lid 118 and the sidewall 110, or may include a latch, strap, buckle, threaded connection, or other mechanical locking feature. In certain configurations, the lid 118 may be rotationally fixed relative to the container 102 when the lid 118 is secured in place. In some embodiments, the inlet 104 may extend through the lid 118. In other embodiments, the inlet 104 may extend through the sidewall 110.

The processor 100 may include an irrigation port 120 configured to introduce fluids into the container 102. For example, saline, antibiotics, or other fluids may be introduced through the irrigation port 120 during processing. In some embodiments, the irrigation port 120 may extend through the lid 118. In other embodiments, the irrigation port 120 may extend through the sidewall 110 or the bottom wall 112.

The processor 100 may include a core 124 disposed within the container 102. The core 124 may also be referred to as a rotor. The core 124 may be positioned within the chamber 108 and may be rotatable relative to the container 102. Rotation of the core 124 may impart rotational movement to the biological material within the chamber 108, thereby generating centrifugal forces within the biological material. The centrifugal forces may facilitate separation of different components of the biological material. For example, rotation may cause oil, blood, aqueous fluids, or other materials to separate from adipose tissue.

The core 124 may be rotatable about an axis A-A. The axis A-A may extend vertically through a center of the container 102. The axis A-A may extend through the outlet 106. The axis A-A may extend through the core 124. The axis A-A may be coaxial with a central axis of the core 124.

In certain embodiments, the processor 100 may be configured such that rotation of the core 124 occurs while one or more fluid ports remain in fluid communication with external tubing, thereby permitting biological material to flow into and out of the chamber 108 during operation of the processor 100.

The processor 100 may include an actuator 128 configured to rotate at least one of the core 124 and the container 102. In some embodiments, the actuator 128 may rotate the core 124 relative to the container 102. In other embodiments, the actuator 128 may rotate the container 102 relative to the core 124. In still other embodiments, the actuator 128 may rotate both the core 124 and the container 102 either simultaneously or at separate intervals, and at the same or different rotational speeds.

The actuator 128 may comprise a push-to-spin mechanism. The actuator 128 may include an activator 130 configured to receive manual or automated input. For example, the activator 130 may comprise a push button movable along a generally linear path. The actuator 128 may include a motion conversion mechanism configured to convert linear movement of the activator 130 into rotational movement of the core 124. The motion conversion mechanism may include cams, ratchets, gears, or other motion conversion components.

The actuator 128 may comprise a powered device configured to generate motion for rotating the core 124. For example, the actuator 128 may include an electric motor, servo motor, stepper motor, brushless motor, or other powered drive mechanism capable of generating rotational or linear motion. In some examples, the actuator 128 may directly rotate the core 124 through a drive shaft or coupling. In other examples, the actuator 128 may rotate an intermediate component, such as a gear train, belt drive, friction wheel, or other transmission element that transfers rotational motion to the core 124. The actuator 128 may include an internal power source such as one or more batteries. The batteries may be rechargeable or disposable. In other configurations, the actuator 128 may be configured to receive power from an external source, such as a wall outlet, medical equipment power supply, or other external power system. The actuator 128 may therefore include a power interface, such as a power cord, connector, charging port, or wireless charging interface configured to supply electrical power to the actuator.

The actuator 128 may further include control circuitry configured to regulate operation of the powered device. For example, the actuator 128 may include one or more switches, buttons, touch controls, or electronic interfaces configured to allow a user to start, stop, or adjust operation of the actuator. The actuator 128 may also include speed control components configured to vary the rotational speed of the core 124. In some examples, the actuator 128 may include programmable control logic configured to control rotational speed, acceleration, deceleration, or operational timing of the core 124. Sensors such as rotational speed sensors, load sensors, or position sensors may be included to monitor operation of the actuator and provide feedback to the control circuitry.

Repeated activation of the activator 130 may cause repeated rotation of the core 124 about the rotational axis A-A. Repeated activation of the activator 130 may cause the core 124 to spin at a sufficient rotational speed to generate centrifugal forces required to separate adipose tissue from oil or other biological components contained within the container 102. The actuator 128 may be configured to accelerate the core 124 to a predetermined rotational speed and maintain the rotational speed for a selected period of time to promote separation of biological material components. In some examples, the core 124 may rotate at speeds between approximately 50 RPM and 5,000 RPM. In other examples, the rotational speed may range between approximately 200 RPM and 2,000 RPM depending on the viscosity of the biological material and the desired separation characteristics.

The actuator 128 may include a housing 134 with an engagement member 132 extending from the housing 134. The engagement member 132 may be configured to allow a user to stabilize or grip the actuator 128 while operating the activator 130. For example, a user may engage the engagement member 132 with one or more fingers, such as the index finger and middle finger, while actuating the activator 130 with the thumb. The engagement member 132 may include textured surfaces, ridges, grooves, or ergonomic contours configured to improve grip and stability during operation of the actuator 128.

The engagement member 132 may include a flange, collar, ring, finger rest, or one or more protrusions extending outward from the housing 134. In some configurations, the engagement member 132 may extend circumferentially around at least a portion of the housing 134 to form a generally annular support surface. In other configurations, the engagement member 132 may include one or more radially extending projections spaced around the housing to provide engagement surfaces for a user. The engagement member 132 may be integrally formed with the housing 134 or may be provided as a separate component coupled to the housing 134.

At least one of the actuator 128 and the core 124 may extend through an opening in the lid 118. The housing 134 may be rotationally fixed relative to the lid 118. The actuator 128 may be positioned outside of the chamber 108 while the core 124 is positioned within the chamber 108. The housing 134 may be rotationally fixed relative to the lid 118 such that operation of the actuator causes rotation of the core 124 without rotating the lid 118 or container 102 in certain embodiments.

The core 124 may include a central passage 126 in communication with the outlet 106 such that liquid or solid material from the chamber 108 can move through the central passage 126 and out of the container 102. The central passage 126 may extend along the rotational axis A-A. In some examples, the central passage 126 may be sized to allow adipose tissue, fluids, or other biological materials to pass through the core 124 during operation of the processor 100.

The processor 100 may include an arm 136 coupled to the core 124. The arm 136 may be an impeller arm or tissue-engaging arm. The combined core 124 and arm 136 may form an impeller configured to agitate biological material during operation. The arm 136 may be configured to agitate, mix, or mechanically interact with biological material as the core 124 rotates relative to the container 102. Rotation of the arm 136 may mechanically disrupt aggregates of adipose tissue and fibrous material, thereby promoting separation of adipose tissue clusters and improving filtration efficiency. In some examples, the arm 136 may also be configured to capture or retain fibrous material within the biological material during rotation of the core 124.

The arm 136 may include one or more features to increase its effectiveness at removing impurities from the biological material. For example, the arm 136 may include a base 138 and a plurality of projections 140 extending from the base 138. The plurality of projections 140 may be spaced from each other along a length of the arm 136. The spacing between the projections 140 may be selected based on characteristics of fibrous tissue to be removed. In some examples, the projections 140 may be spaced evenly along the length of the base 138. In other examples, the spacing between projections may vary along the length of the base 138. The projections 140 may extend from the base 138 in a first direction transverse to a longitudinal axis of the base 138. The arm 136 may therefore have a comb-like structure. The projections 140 may extend from the base 138 in different directions. In other examples, the projections 140 may extend around the base about the longitudinal axis of the base 138 such that the arm 136 is shaped like a honey dipper. In some examples, the arm 136 may include absorbent or textured materials configured to capture debris, fibrous tissue, or other unwanted materials during processing.

The arm 136 may be movable from a retracted configuration to an extended configuration during rotation of the core 124. The arm 136 may be coupled to the core 124 by a hinge, living hinge, ball-and-socket joint, flexible joint, or other movable coupling. The arm 136 may be operable to pivot relative to the core 124 about an axis B from a first position P1 to a second position P2. In some examples, centrifugal force generated by rotation or acceleration of the core 124 may cause the arm 136 to pivot outward from the first position P1 toward the second position P2. The arm 136 may extend radially toward a filter 142 as the arm moves toward the second position P2. The centrifugal forces generated during rotation of the core 124 may automatically deploy the arm from the retracted configuration to the extended configuration without requiring a separate actuator.

The arm 136 may include a first end and a second end opposite the first end. The first end may be pivotally coupled to the core 124. The second end may be a free end. The second end may move outward, upward, or radially away from the core 124 as the arm 136 moves from the first position P1 to the second position P2. In some examples, the second end of the arm 136 may be spaced from the core 124 when the arm 136 is in the first position P1. In other examples, the second end of the arm 136 may be adjacent to or in contact with the core 124 when the arm 136 is in the first position P1.

The outward movement of the arm 136 may increase the effective diameter of the rotating structure within the chamber 108 during operation of the processor 100. The arm 136 may therefore engage or interact with biological material at different radial positions within the chamber 108 during operation. The arm 136 may be a centrifugally deployable tissue-engaging arm.

The arm 136 may retract toward the first position P1 when the core 124 decelerates or stops rotating. In some examples, the arm 136 may fully return to the first position P1 when the core 124 stops rotating. The arm 136 may therefore move through multiple cycles of extension and retraction as the core 124 accelerates and decelerates during operation of the processor 100.

The arm 136 may be rotationally fixed relative to the core 124 about a first axis. The first axis may be axis A-A. The arm 136 may be pivotable or rotatable relative to the core about a second axis. The second axis may be axis B. The second axis may be offset from the first axis. The second axis may be transverse to the first axis. The second axis may be perpendicular to the first axis. The arm 136 may rotate about the first axis and the second axis simultaneously.

The processor 100 may include one arm 136, two arms 136, three arms 136, four arms 136, or more than four arms 136 as desired. Each arm may be identical to the other arms. Alternatively, one or more arms may include features different from other arms. In examples with two arms 136, the arms may move toward and away from each other as the core 124 accelerates and decelerates. In examples with a plurality of arms 136, the arms may be spaced about a perimeter of the core 124. The arms 136 may be evenly spaced about the core 124.

The processor 100 may include a filter 142 disposed within the chamber 108 to separate liquid or oil components from the biological material. The filter 142 may be positioned between the core 124 and the sidewall 110 of the container 102. The filter 142 may be spaced from the sidewall 110. The filter 142 may be laterally spaced from each of the sidewall 110 and the core 124. The filter 142 may comprise a mesh, screen, porous structure, or other filtration structure. In some examples, the mesh may include openings between approximately 10 micrometers and 500 micrometers. The filter 142 may therefore allow fluids such as oil or blood to pass while retaining adipose tissue.

The filter 142 may be a flexible element. The filter 142 may change shape during operation of the processor 100. The filter 142 may have a shape that correlates with the movement pattern of the one or more arms 136 as the arm 136 moves between the first and second positions P1 and P2. For example, the filter 142 may be spaced from the sidewall 110 when the arm 136 is in the first position P1. The filter 142 may move toward the sidewall 110 as the arm 136 moves toward the second position P2. At least a portion of the arm 136 may contact, displace, or compress the filter 142 as the arm moves toward the second position P2. Such contact, displacement, or compression may urge separated liquid components toward a collection region of the chamber 108.

The opening 123 of the outlet 106 may extend through the filter 142 as well as one of the bottom wall 112 and sidewall 110 such that solid material can exit the chamber 108 through the outlet 106.

The filter 142 may contact the sidewall 110 when the arm 136 is in the second position P2. The filter 142 may be spaced from the bottom wall 112 when the arm 136 is in at least one of the first and second positions P1 and P2 to define a flow region 117 between the filter 142 and at least one of the bottom wall 112 and the sidewall 110.

The second outlet 114 may be configured to remove material from the flow region 117. The opening 115 of the second outlet 114 may be positioned on the bottom wall 112 such that liquid components or small solid components between the filter 142 and the bottom wall 112 are directed through opening 115 and out of the second outlet 114. In some examples, deformation of the filter 142 in response to movement of the arm 136 may actively promote migration of liquid components toward the second outlet 114.

The inlet 104 may be coupled to an aspirator, liposuction device, syringe, pump, or other aspiration device such that biological material may be aspirated from a patient and introduced into the chamber 108. The inlet 104 may be connected to the aspiration device through tubing, conduit, or other fluid connectors. The outlet 106 may be coupled to a pump, syringe, vacuum source, or other transfer device through a conduit 116, which may comprise tubing, piping, or other fluid conduits. In certain embodiments, the processor 100 may operate under negative pressure generated by a pump or vacuum source coupled to one or more outlets. The negative pressure may assist in drawing separated liquid fractions, such as oil, blood, or aqueous fluids, through the filter while retaining adipose tissue or other desired biological material within the chamber 108.

In some examples, the conduit 116 may be detachably coupled to the outlet 106 to facilitate connection or removal of external fluid transfer components. The processor 100 may also include one or more caps, plugs, valves, or other flow-control elements configured to selectively seal or regulate flow through the inlet 104, outlet 106, or second outlet 114 when not in use.

The second outlet 114 may be detachably coupled to a conduit or tubing connected to a receptacle such as a trap cannister. The trap cannister may collect separated liquid components including oil and aqueous portions of the biological material. The receptacle may be coupled to a vacuum source that provides negative pressure through the receptacle, the conduit or tubing, and the second outlet 114 to draw liquid material from the chamber 108.

Interaction between the flexible filter 142 and the one or more arms 136 may enhance separation efficiency of the biological material. For example, deformation of the filter 142 in response to movement of the arms 136 may reduce clogging of the filter, increase effective surface area available for filtration, and promote directed movement of liquid components toward the second outlet 114. Additionally, repeated contact between the arms 136 and the filter 142 during rotation of the core 124 may facilitate mechanical disruption of fibrous material and improve release of entrained fluids, thereby improving overall separation performance.

The processor 100 may be part of a closed-loop system in which biological material is aspirated from a patient, processed within the chamber 108, and returned to the patient. For example, biological material may enter the chamber 108 through the inlet 104, centrifugal forces generated by rotation of the core 124 may separate oil or other fluids from adipose tissue. The separated fluids may exit the container 102 through the second outlet 114. Processed adipose tissue may exit the container 102 through the outlet 106 and conduit 116 for reinjection into the patient. In certain embodiments, biological material may continue to flow into and out of the chamber 108 while the core 124 rotates such that one or more of aspiration of biological material, centrifugal processing of the biological material, reinjection of processed adipose tissue, and removal of fluid may occur simultaneously during operation of the processor 100.

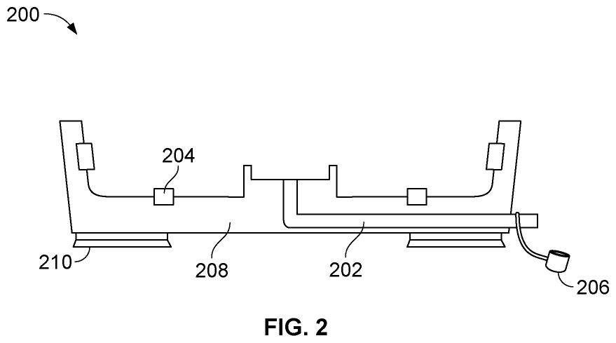

Referring to FIG. 2, a base 200 is shown. The base 200 may be configured to receive a portion of the container 102. The base 200 may have a body 208 that defines a recess such that the container 102 can be set into the recess. In other examples, the base 200 may be generally planar and the container 102 may sit on an upper surface of the base 200. The container 102 may be detachably coupled to the base 200.

The container 102 may be rotatable relative to the base 200. In some examples, the base 200 may be operable to rotate the container 102 relative to the base 200. In other examples, the actuator 128 may cause rotation of the container 102 relative to the base 200. For example, the core 124 may be rotationally fixed relative to the container 102 such that the actuator 124 rotates the core 124 and container 102 simultaneously.

The base 200 may include a conduit 202 configured to connect to outlet 106 when the container 102 is coupled to the base 200. The conduit 202 may be sized and shaped to receive at least a portion of the outlet 106. The base 200 may include a cap 206 configured to selectively seal the conduit 202. In some examples, conduit 116 is removed from outlet 106 prior to coupling the container 102 to the base 200. In other examples, the conduit 116 can be routed through the base 200.

The base 200 may include one or more low-friction elements 204 configured to facilitate rotation of the container 102 when the container is coupled to the base. The low-friction element 204 may be a bearing, roller, bushing, or similar mechanical element. The low-friction elements 204 may contact a lower surface of the container 102. In other examples, the low-friction elements 204 may contact the sidewall 110 and the lower surface of the container 102.

The base 200 may include one or more anchoring elements 210 configured to resist movement of the base 200 when forces are generated during operation. The anchoring elements 210 may resist forces generated when rotating the container 102 relative to the base 200 or when the core 124 rotates relative to the container 102. The anchoring element 210 may comprise a suction cup, high-friction material, weighted structure, or other stabilizing component.

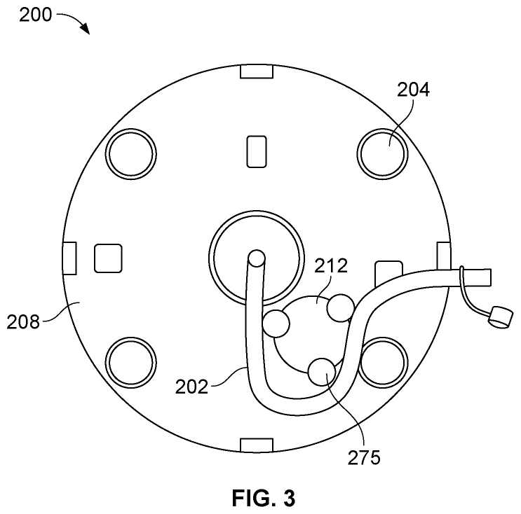

Referring to FIG. 3, the base 200 may include a motor 212. In some examples, the motor 212 is configured to rotate the container 102 about axis A-A. In other examples, the motor 212 is configured to drive movement of biological material through the conduit 202. The motor 212 may be configured to simultaneously rotate the container 102 and drive movement of biological material through the conduit 202. The motor 212 may be a stepper motor, servo motor, or other powered rotary device.

In embodiments in which the motor 212 rotates the container 102, the motor 212 may be coupled to one or more rotational elements configured to engage the container 102 and impart rotational motion. The rotational element may include one or more rollers, wheels, friction surfaces, or similar element that contacts an external surface of the container 102. The rotational element may be a gear that is coupled with a geared surface on the container 102 to rotationally link the container 102 to the motor 212. In other examples, the rotational element comprises a gear configured to engage a corresponding geared surface on the container 102 to rotationally link the container 102 to the motor 212. The motor 212 may thereby be operable to rotate the container 102 about the axis A-A.

In embodiments in which the motor 212 is configured to move biological material through the conduit 202, the motor 212 may be coupled to a roller 275 that engages the conduit 202. The conduit 202 may comprise a flexible material such that rotation of the roller 275 compresses and releases the conduit 202 to drive movement of biological material therethrough. In this manner, the motor 212 and roller 275 may function as a peristaltic pumping mechanism. The speed of the motor 212 may be adjustable such that a flow rate of biological material through the conduit 202 is controllable. The roller 275 may simultaneously contact the conduit 202 and an external surface of the container 102 to simultaneously rotate the container 102 and drive movement of the biological material through the conduit 202.

The processor 100 may therefore be configured to operate in multiple rotational modes. In some embodiments, the container 102 rotates relative to the base 200 while the core 124 remains rotationally fixed. In other embodiments, the core 124 rotates relative to the container 102 while the container 102 remains rotationally fixed. In still other embodiments, both the container 102 and the core 124 rotate simultaneously. The rotational speeds of the container 102 and the core 124 may be the same or different when rotating simultaneously. The core 124 and the container 102 may rotate in different directions.

In certain embodiments, the container 102 may be configured as a disposable component intended for single-patient use. For example, the container 102 may be provided as a sterile component that is discarded after completion of a medical procedure. In contrast, the base 200 may be configured as a reusable component capable of being used with multiple containers 102. The base 200 may therefore be configured to detachably receive and support successive containers during different procedures. In some examples, the base 200 may include mechanical interfaces, alignment features, electrical connectors, drive couplings, or fluid connections that allow the base 200 to operably couple with each container 102 while allowing the container to be removed and replaced after use. The container 102 may therefore function as a disposable biological processing cartridge configured to be removably coupled to the base 200. The container may include the chamber, filter, core, and fluid ports, while the base may include the motor, drive mechanism, and control electronics.

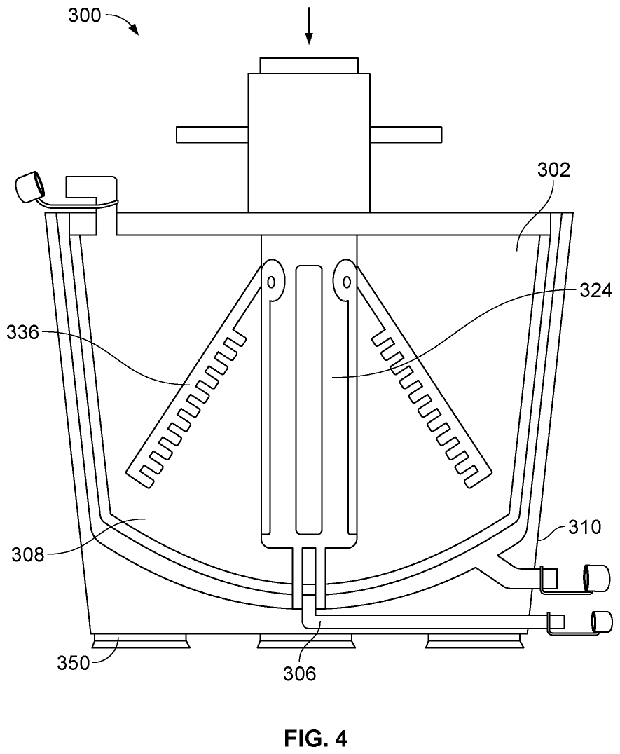

Now with reference to FIGS. 4 to 7, alternative constructions of the processor 300, 500, core 424, and base 600 are illustrated. It should be appreciated that the processor 300, 500, the core 424, and the base 600 are similar to the processor 100, core 124, and base 200, respectively. Only the differences between the processor 300, 500 and processor 100, the differences between the core 424 and core 124, and the differences between the base 600 and base 200 are discussed in detail herein. Further, like components are given a like reference number plus “300”; “400”; “500”; and “600” respectively. Unless otherwise stated, the structure and operation of these corresponding components may be similar to, or the same as, those previously described.

Referring to FIG. 4, the processor 300 may include one or more anchoring elements 350 configured to resist movement of the container 302 when forces are generated during operation of the processor. For example, rotation of the container 302 or rotation of the core 324 relative to the container 302 may generate forces that could otherwise cause the processor 300 to move relative to a supporting surface. The anchoring elements 350 may therefore stabilize the processor during operation. In some examples, the anchoring elements 350 may comprise suction cups configured to adhere to a supporting surface. In other examples, the anchoring elements 350 may include high-friction pads, weighted structures, clamps, adhesive surfaces, or other stabilizing components capable of resisting movement of the processor 300.

The processor 300 may include one or more arms 336 coupled to the core 324. The arms 336 may be rotationally fixed relative to the core 324 such that the arms rotate with the core during operation. In some embodiments, the arms 336 may be oriented at different angles relative to the longitudinal axis of the core 324. For example, one arm 336 may be oriented at an angle configured to contact biological material toward a lower portion of the chamber 308 during rotation, while another arm 336 may be oriented at a different angle configured to contact biological material toward an upper portion of the chamber 308. The different orientations of the arms 336 may promote mixing, agitation, and separation of biological material within the chamber 308 during operation of the processor.

The outlet 306 may extend through a bottom of the chamber 308 and may then exit through the sidewall 310 of the container 302. Routing the outlet 306 through the sidewall 310 may allow the processor 300 to rest on a table, tray, or other supporting surface without interference from tubing or fluid connections extending from the bottom of the container 302. This configuration may improve stability of the processor during operation and may also facilitate connection of tubing to external pumps, syringes, or other medical devices.

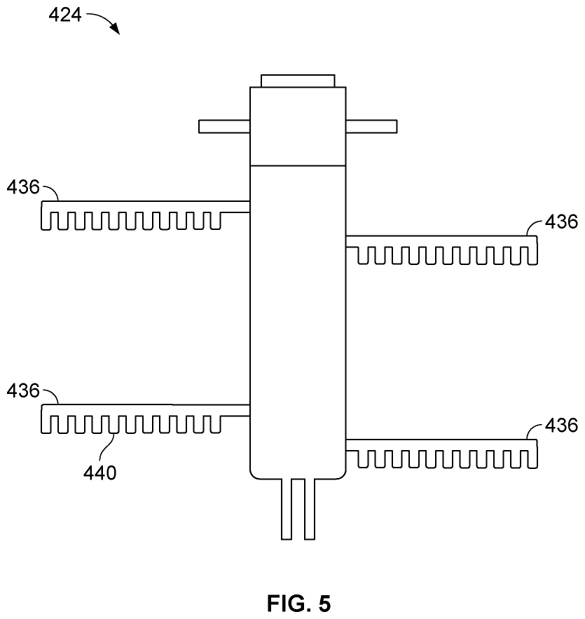

Referring to FIG. 5, a core 424 is illustrated that may include a plurality of arms 436. The arms 436 may be rotationally fixed relative to the core 424 such that the arms rotate together with the core during operation. The arms 436 may be positioned at different axial positions or heights along the length of the core 424. Positioning the arms 436 at different heights may allow biological material to be engaged and agitated at different vertical regions within the chamber during rotation of the core 424.

The arms 436 may also be spaced circumferentially about the core 424. In some embodiments, the arms 436 may be evenly spaced around the perimeter of the core 424. In other embodiments, the arms 436 may be spaced at non-uniform circumferential intervals to create different flow patterns within the chamber during rotation.

Each arm 436 may be manufactured from the same material. For example, the arms 436 may be formed from polymer materials, metal materials, composite materials, or other suitable materials capable of withstanding rotational forces during operation. In some embodiments, each arm 436 may include the same pattern of projections 440 extending from the arm. In other embodiments, at least one arm 436 may include a pattern of projections 440 that differs from the pattern of projections on another arm. Varying the patterns of projections may improve the ability of the arms 436 to capture fibrous tissue, break apart aggregates, or otherwise interact with biological material during processing.

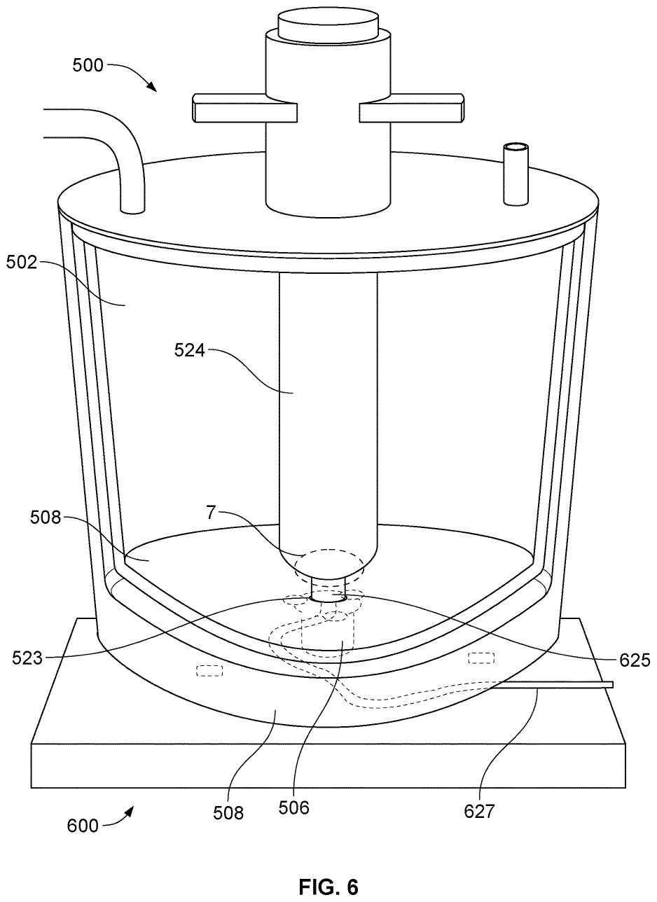

Referring to FIG. 6, a processor 500 may be coupled to a base 600. The base 600 may be configured to couple external tubing to the container 502 while the container 502 rotates relative to the base 600. In this configuration, biological material may flow into and out of the container 502 while the processor 500 is operating. Such an arrangement may allow biological material to be processed while the processed biological material is simultaneously delivered to a patient or transferred to another medical device. The processor 500 may be detachably coupled to the base 600 such that the container 502 may be removed and replaced after a procedure. In other examples, the processor 500 may be fixed to the base 600 while remaining rotatable relative to the base 600.

An opening 523 may extend through the filter 542 and the bottom wall 512 of the container 502. The opening 523 may be in fluid communication with the outlet 506 such that material within the chamber 508 may flow through the opening 523 and through the outlet 506. In some embodiments, the opening 523 may be aligned with a central axis of the container 502 or the core 524.

The base 600 may include a channel 627 in fluid communication with the outlet 506. The channel 627 may be configured to receive or couple with external tubing, conduit, or other fluid transfer components. The base 600 may include a locking element configured to secure the external tubing to the base 600. In certain embodiments, the external tubing may be rotationally fixed relative to the base 600 while the container 502 rotates relative to the external tubing.

An adapter 625 may be configured to couple with the core 524. The adapter 625 may comprise a rigid port adapter configured to mechanically support the core 524 and to maintain fluid communication between the chamber 508 and the channel 627. In some examples, the adapter 625 may extend through the opening 523 to couple to the core 524. In other examples, the opening 523 may be sized to allow a portion of the core 524 to extend through the opening 523 to couple to the adapter 625. The adapter 625 may be fixed relative to the base 600. At least a portion of the adapter 625 may be positioned within the outlet 506. The adapter 625 may therefore remain rotationally fixed relative to the base 600 while the container 502 rotates relative to the base 600. One of the adapter 625 and the core 524 may be rotatable relative to the other of the adapter 625 and the core 524.

The processor 500 may include a rotational interface configured to maintain fluid communication between the chamber 508 and one or more stationary conduits while at least one internal component of the processor 500 rotates relative to the conduits. The rotational interface may include a rotary fluid coupling, rotary seal assembly, rotating union, or similar structure that allows biological material to pass between rotating and non-rotating components. This configuration may permit biological material to flow continuously into and out of the processing chamber while centrifugal forces are generated within the chamber.

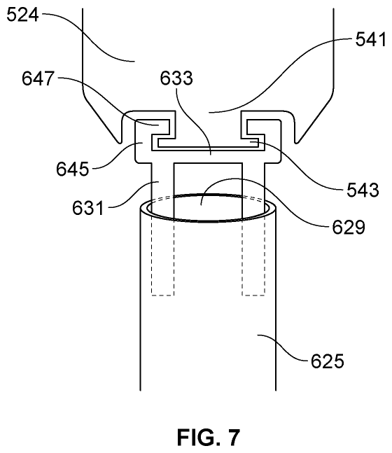

Referring to FIG. 7, a coupling 631 may be coupled to the core 524 and the adapter 625. The coupling 631 may be configured to transmit mechanical support between the core 524 and the adapter 625 while allowing relative rotation between the core 524 and adapter 625. In some embodiments, the coupling 631 may be rotationally fixed relative to one of the core 524 and the adapter 625 while being rotatable relative to the other. In some examples, the core 524 may be rotatable relative to the coupling 631 and the coupling 631 may be rotatable relative to the adapter 625.

The core 524 may include an engagement feature configured to engage a corresponding feature of the coupling 631. For example, the core 524 may include a protrusion 541 extending from the core 524. A flange 543 may extend from the protrusion 541. The protrusion 541 may extend longitudinally while the flange 543 may extend radially or laterally from the protrusion 541.

The coupling 631 may include an engagement feature configured to receive the engagement feature of the core 524. For example, the coupling 631 may include a first wall 645 defining a recess configured to receive at least a portion of the protrusion 541 and the flange 543. The coupling 631 may also include a second wall 647 at least partially enclosing the recess. The first wall 645 may prevent lateral or radial movement of the protrusion 541 during operation. The second wall 647 may prevent longitudinal movement of the protrusion 541 or flange 543. In some examples, the first wall 645 may form a sidewall of the recess and the second wall 647 may form an upper wall of the recess.

The adapter 625 may include an opening 629 that is in fluid communication with the chamber 508 and the channel 627. The opening 629 may be configured to receive at least a portion of the coupling 631. The coupling 631 may include one or more prongs 633 configured to extend into the opening 629 of the adapter 625. The prongs 633 may assist in stabilizing the coupling 631 and may limit lateral or radial movement of the core 524 relative to the adapter 625 during operation of the processor 500.

In certain embodiments, the processor 500 may be configured such that one or more fluid conduits remain rotationally fixed relative to the base 600 while the container 502 rotates relative to the base 600. For example, external tubing connected to the channel 627 may remain stationary while the container 502 rotates during operation of the processor 500. The adapter 625, coupling 631, and associated components may permit fluid communication between the chamber 508 and the stationary external tubing while accommodating relative rotation between the container 502 and the base 600. This configuration may allow biological material to enter and exit the chamber 508 during rotation of the container 502.

In some embodiments, the processor 500 may operate as part of a continuous flow loop configured to circulate biological material through the processor during operation. For example, biological material may be aspirated from a patient through a first conduit and introduced into the chamber 508 through the inlet 504. The biological material may then be processed within the chamber 508 while the container 502 rotates and centrifugal forces act on the biological material. Processed adipose tissue may exit the chamber 508 through the outlet 506 and may be delivered through external tubing to a reinjection device for return to the patient. In certain examples, the continuous flow loop may therefore allow aspiration of biological material, centrifugal processing of the biological material, and reinjection of processed biological material to occur simultaneously. The processor 500 may include a fluid interface configured to maintain fluid communication between the chamber 508 and one or more stationary conduits while at least one of the container 502 and the core 524 rotates relative to the stationary conduits.

Aspects of the present disclosure are not limited to the exemplary structural details and component arrangements described in this description and shown in the accompanying drawings. Many aspects of this disclosure may be applicable to other aspects and/or capable of being practiced or carried out in various variants of use, including the examples described herein. It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Several different reference axes are described, including a lateral axis X-X, a longitudinal axis, Y-Y, and/or a vertical axis Z-Z oriented about a datum to form a three-dimensional Cartesian coordinate system. Relevant arrangements may be described in relation to different reference axes. For example, the longitudinal axis may be non-parallel with the lateral axis in some perspectives, meaning that one axis extends across the other; and the vertical axis may be non-parallel with the lateral and longitudinal axis in some perspectives, meaning that all three axes extend across one another. Relative terms such as “long” and “elongated” may describe any aspect having a length along one reference axis (e.g., the longitudinal or vertical axis) that is longer in relation to a width along a non-parallel reference axis (e.g., the lateral axis). As utilized herein, these terms are provided for convenience and not intended to limit this disclosure unless claimed.

The terms “proximal” and “distal” may be used to describe some structures in relation to a reference axis. As utilized herein, these terms are provided for convenience to orient the reader and not intended to limit this disclosure unless claimed. Movements and forces may be similarly described in relation to any reference axis. Here again, descriptions of certain movements and forces in relation to certain reference axes are provided to help explain certain inventive aspects by way of example and are not intended to limit this disclosure unless claimed.

Unless specifically set forth herein, the terms “a”, “an” and “the” are not limited to one element but instead should be read as meaning “at least one”. Inclusive terms such as “comprises,” “comprising,” “includes,” “including,” and variations thereof, are intended to cover a non-exclusive inclusion, such that aspects of any apparatus, kit, method, and system described herein, or element(s) thereof described as comprising a list of elements does not include only those elements but may include other elements not expressly listed and/or inherent thereto. Unless stated otherwise, the term “exemplary” means “example” rather than “ideal.” Various terms of approximation may be used, including “approximately” and “generally.” Approximately means “roughly” or within 10% of a stated number or outcome and generally means “usually” or more than a 50% probability of a stated number or outcome.

Connective terms such as “coupled to,” “coupled with,” and “coupling” are intended to generically describe a structural connection between two or more elements. Some structural connections may be “fixedly coupled” so that the connected elements are generally non-rotatable or non-movable relative to one another, as when the elements are formed together (e.g., cast, bolted, and/or welded) and cannot be rotated independently without deflecting relative to one another or being damaged. Other structural connections may be “rotatably or movably coupled” so that the connected elements are coupled together to permit movements relative to one another, as when the elements are pinned together (e.g., with any type of rotating, sliding, and/or telescoping connection) and can be rotated or moved freely and independently without damage. Unless stated otherwise, these exemplary connective terms and their modifiers may comprise any such variations.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. The words “right”, “left”, “lower” and “upper” designate directions in the drawings to which reference is made. The words “inwardly” and “outwardly” refer to directions toward and away from, respectively, the geometric center of the device.

To the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

I/We claim:

1. A biological material processing device comprising:
a container configured to receive biological material;
an inlet port configured to receive the biological material into the container;
a core disposed within the container and rotatable relative to the container;
an actuator configured to rotate the core; and
an arm coupled to the core, the arm movable from a retracted configuration to an extended configuration during rotation of the core, the arm configured to capture fibrous material within the biological material during rotation of the core.

2. The biological material processing device of claim 1, wherein at least a portion of the arm extends outward from a first position to a second position during rotation of the core.

3. The biological material processing device of claim 2, wherein the arm retracts to the first position as the core decelerates.

4. The biological material processing device of claim 2, wherein the core includes a recess, at least a portion of the arm is in the recess in the first position, and the portion of the arm is out of the recess in the second position.

5. The biological material processing device of claim 1, wherein the arm is rotationally fixed relative to the core about a first axis.

6. The biological material processing device of claim 5, wherein the arm is rotatable relative to the core about a second axis offset from the first axis.

7. The biological material processing device of claim 1, wherein the arm comprises a base and a plurality of projections extending from the base, the projections spaced from each other along the base, the projections configured to capture fibrous strands within the biological material.

8. The biological material processing device of claim 1, wherein the arm is connected to the core by a hinge.

9. The biological material processing device of claim 1, further comprising a filter within the container, the filter positioned between the core and a sidewall of the container, the filter configured to separate liquid or oil components from the biological material.

10. The biological material processing device of claim 9, wherein the filter comprises a mesh having openings between approximately 10 micrometers and 500 micrometers.

11. The biological material processing device of claim 9, wherein the filter is flexible and at least one of the arm and the biological material contact the filter during rotation of the core thereby causing the filter to flex from a first configuration to a second configuration.

12. The biological material processing device of claim 9, wherein the filter separates oil from adipose tissue.

13. The biological material processing device of claim 1, wherein the rotation of the core processes the biological material to separate solid components from liquid components, and
wherein the processing device further comprises a first outlet for removing the solid components from the container.

14. The biological material processing device of claim 13, further comprising a second outlet for removing the liquid components from the container.

15. The biological material processing device of claim 13, further comprising a valve configured to selectively prevent flow of material through the first outlet.

16. The biological material processing device of claim 13, further comprising tubing connected to the first outlet for transferring the solid component to a patient.

17. The biological material processing device of claim 13, wherein the solid component comprises fat.

18. The biological material processing device of claim 1, further comprising a removable lid detachably coupled to the container, the lid forming a liquid tight seal with the container when the lid is coupled to the container.

19. The biological material processing device of claim 18, further comprising a locking mechanism that secures the lid to the container to maintain sterility of the container when the lid is coupled to the container.

20. The biological material processing device of claim 1, further comprising an irrigation port for introducing saline, antibiotics, fluids, or solids into the container.

21. The biological material processing device of claim 1, wherein the actuator is manually actuated.

22. The processing device of claim 21, wherein the actuator comprises a push-to-spin mechanism.

23. The biological material processing device of claim 1, further comprising a base coupleable with the container, wherein the container is rotatable relative to the base.

24. The biological material processing device of claim 23, wherein the actuator causes rotation of the core while the container rotates relative to the base.

25. The biological material processing device of claim 1, wherein the container comprises a rotating biological processing chamber configured to maintain fluid communication with at least one stationary conduit during rotation.

26. The biological material processing device of claim 23, further comprising a motor that rotates the container relative to the base.

27. The biological material processing device of claim 1, further comprising a base coupleable with the container, the base including a motor; and
an outlet conduit coupled to the container such that biological material moves out of the container through the conduit,
wherein motor drives movement of the biological material through the conduit.

28. The biological material processing device of claim 27, further comprising a peristaltic pump that includes the motor.

29. The biological material processing device of claim 1, further comprising an outlet conduit in fluid communication with the container and a patient,
wherein the inlet port is coupled to a liposuction device having an end within the patient such that the biological material circulates in a continuous closed loop between the patient, the container, and back to the patient during rotation of the core.

30. The biological material processing device of claim 1, wherein the arm comprises absorbent material configured to capture debris.

31. A tissue processing system comprising:
a container configured to receive biological material;
a core disposed within the container;
a first conduit configured to deliver biological material from a patient to the container;
a second conduit configured to return biological material from the container to the patient; and
a spinning mechanism configured to rotate at least one component of the tissue processing system,
wherein biological material circulates in a continuous closed loop between the patient and the container during rotation of the at least one component of the tissue processing system.

32. The system of claim 31, further comprising a base configured to support the container.

33. The system of claim 32, wherein the base includes a motor to rotate the container relative to the base.

34. The system of claim 32, wherein the base includes rollers to support the container and allow the container to rotate relative to the base.

35. The system of claim 31, wherein the container remains stationary while the core rotates.

36. The system of claim 31, wherein the system is configured to operate in a first mode in which the container rotates.

37. The system of claim 31, wherein the system is configured to operate in a second mode in which the core rotates.

38. The system of claim 31, wherein the system is configured to sequentially operate in a first mode in which the container rotates and then a second mode in which the core rotates relative to the container.

39. The system of claim 31, wherein biological material is processed while circulating between the patient and the container.

40. The system of claim 39, wherein aspiration of biological material and reinjection or processed biological material occur simultaneously.

41. The system of claim 31, further comprising an arm coupled to the core wherein the arm captures fibrous tissue during rotation.

42. The system of claim 41, wherein at least a portion of the arm extends outward from a first position to a second position during rotation of the core and retracts to the first position as the arm decelerates.

43. The system of claim 42, wherein the arm is rotationally fixed relative to the core about a first axis, and
wherein the arm is rotatable relative to the core about a second axis relative to the core as the arm moves from the first position to the second position.

44. The system of claim 43, wherein the arm rotates about the first axis and the second axis simultaneously.

45. The system of claim 31, wherein the system is configured for fat grafting procedures.

46. A biological material processing device comprising:
a container configured to receive biological material;
a core disposed within the container;
a fluid port configured to communicate biological material between the container and an external conduit; and
a base configured to secure the external conduit,
wherein the core rotates relative to the fluid port while biological material flows through the fluid port.

47. The device of claim 46, wherein the base holds the external conduit in a fixed position.

48. The device of claim 46, wherein the core rotates relative to the base.

49. The device of claim 46, wherein the external conduit remains stationary while the core rotates.

50. The device of claim 46, wherein the fluid port remains fixed relative to external conduit.

51. The device of claim 46, wherein the base comprises a rigid port adapter.

52. The device of claim 46, wherein the base is configured to prevent rotation of the external conduit.

53. The device of claim 46, wherein the core spins about an axis and the axis extends through at least a portion of the fluid port.

54. The device of claim 46, wherein the core includes deployable arms.

55. A method of processing biological material comprising:
aspirating biological material from a patient into a container of a biological material processing device;
rotating a component of the biological material processing device to separate components of the biological material;
capturing impurities with deployable arms within the container; and
removing processed tissue from the container.

56. The method of claim 55, wherein the capturing impurities step includes capturing fibrous tissue.

57. The method of claim 55, further comprising circulating the biological material in a closed loop.

58. The method of claim 55, wherein biological material processing device includes a core within the container and the rotating step includes rotating the core relative to the container.

59. The method of claim 58, wherein the biological material processing device includes an arm coupled to the core and the method further comprises extending at least a portion of the arm away from the core by centrifugal force.

60. The method of claim 59, further comprising retracting the deployable arms by decelerating rotation of the core.

61. The method of claim 55, wherein biological material processing device includes a core within the container and the rotating step includes rotating the container relative to the core.

62. The method of claim 55, further comprising reinjecting the processed tissue into the patient.

63. The method of claim 62, wherein the aspirating step and the reinjecting step occur simultaneously.

64. The method of claim 55, further comprising filtering oil from adipose tissue.

65. The method of claim 55, further comprising introducing at least one of saline and antibiotics into the container.

66. The method of claim 55, wherein the rotating step includes rotating the component with a motor.

67. The method of claim 55, wherein the rotating step includes rotating the component using a manually activated push-to-spin mechanism.

68. The method of claim 55, further comprising separating oil from adipose tissue using a mesh filter.

69. The method of claim 55, further comprising delivering processed adipose tissue for fat grafting.
